(19) United States
(12) Patent Application Publication
(10) Pub. No.: US 2003/0087436 A1
(43) Pub. Date: May 8, 2003

(54) Process for the production of biologically active polymeric nanoparticle-nucleic acid conjugates

(75) Inventor: Ernst Bayer, Tubingen (DE)

Correspondence Address: FITCH EVEN TABIN AND FLANNERY, 120 SOUTH LA SALLE STREET, SUITE 1600, CHICAGO, IL, 60603-3406, US

(73) Assignee: SKW Trostberg Aktiengesellschaft

(21) Appl. No.: 10/174,617

(22) Filed: Jun. 19, 2002

Related U.S. Application Data
(63) Continuation of application No. 09/295,817, filed on Apr. 21, 1999, now abandoned.
Application No. 09/295,817 is a continuation of application No. PCT/EP97/05790, filed on Oct. 21, 1997.

(30) Foreign Application Priority Data
Oct. 23, 1996 (DE) DE-196 43 738.5

(51) Int. Cl. A61K 48/00; B01J 13/02; B01J 13/04; C12N 15/85; C08G 63/48; C08G 63/91

(52) U.S. Cl. 435/455; 264/4.1; 514/44; 525/54.5

(57) ABSTRACT

A process is disclosed for producing biologically active polymer nanoparticle-nucleic acid conjugates by polymerizing vinyl monomer with a low-water solubility in an aqueous solution, then reacting the resulting polymer suspensions with the nucleic acids. The process is characterized in that the vinyl monomers are emulsion polymerized in the presence of cationic radical starters but in the absence of any emulsifier. The thus obtained polymer nanoparticle-nucleic acid conjugates are sufficiently stable in biological media and are characterized by a high transport efficiency through cellular membranes.

The present invention relates to a process for the production of biologically active polymeric nanoparticle-nucleic acid conjugates and their use for gene transfer and/or for the control of gene expression.

The use of nucleic acids and nucleic acid derivatives, such as for example synthetically produced oligodeoxynucleotides and their derivatives, for controlling gene expression (anti-sense strategy) or of DNA fragments and plasmids for gene transfer require effective transport through cellular membranes. Additionally, the nucleic acids should be protected against enzymatic degradation and reach their target location in the cell (cell nucleus, mRNA in the cytoplasma) in sufficient concentration in order to ensure the desired effect.

Colloidal carrier systems for the transport of biologically and/or therapeutically effective substances have been used for la long time, whereby the following requirements are made of the carrier system:

small particle size which clearly lies below the size of cell structures (diameter smaller than 1 μm)

high loading capacity

low toxicity

the possibility for surface modification

control of the target location by variation of size and/or surface properties

controlled release of the adsorptively bound substances

no side effects as a result of carrier material, degradation products or adjuvents.

The use of synthetically produced polymeric particles in the nm-range for adsorptive binding of nucleic acids is described in FR-A 2, 649, 321 for example. In this connection, poly(alkylcyanoacrylates) are provided with a positive surface charge through adsorptive binding of low molecular weight, hydrophobic cations in order to permit attachment of negatively charged oligodeoxyribonucleotides over ionic interactions. A disadvantage in this system is the fact that the pure adsorptively binding of low molecular weight cations is not very stable and the hydrophobic cations used as well as the degradation products of the polymeric parent substance can have toxic properties.

According to EP-A 430 517, the production of biomosaic polymers in the form of membranes, films or particles by means of emulsion polymerization are described, whereby the polymerization essentially occurs in the presence of a surface active substance (ionic or non-ionic tenside) as well as a biologically active substance (such as for example, nucleic acids) and the biologically active material is irreversibly polymerized into the polymeric parent substance and/or bound on its surface.

Finally, a particular medicament is known from WO 96/24 377 which consists of cationic polymeric nanoparticles and peptides and/or modified or non-modified-nucleic acids. Particle mixed polymers based on acrylic acid, acrylic acid esters as well as methylacrylic acid esters function in this connection as polymeric parent substances. The introduction of the functional cationic groups necessary for binding of the active ingredient occurs through the use of suitably modified comonomers which possess amino functions that are capable of being protonated and/or alkylated. These charge carrying monomer units are reacted in any mixture ratio with the suitable unmodified monomer in a radical or ionic polymerization reaction. Aside from the fact that these monomeric units are complicated to produce, non-homogeneous products frequently arise in the production of these mixed polymers as a result of the differences present in the solubility and reactivity of these monomers.

Therefore, the object of the present invention was to develop a process for the production of polymeric nanoparticle-nucleic acids conjugates which do not have the mentioned disadvantages corresponding to the art, but instead, permit the production of homogeneous polymeric nanoparticle-nucleic acids conjugates in a simple, cost effective and reproducible manner that have sufficient stability in biological media and are distinguished by a high efficiency in the transport through cellular membranes.

The problem is solved according to the invention by carrying out a polymerization of vinyl monomers with a low water solubility in the presence of cationic radical starters in the form of an emulsifier-free emulsion polymerization and subsequently reacting the obtained polymer suspensions with nucleic acids.

It has been surprising shown that the materials produced in this manner are distinguished by a high nucleic acid loading as well as a sufficient stability against enzymatic degradation. Additionally, these conjugates can be varied in their properties by many parameters, such as for example polymeric parent substance, particle size, surface modification, nucleic acid modification, etc., which was also not predictable. The conjugates according to the invention are different from the conjugates according to WO 96/24 377 in that they contain-cationic groups practically exclusively on the chain end of the respective polymer chains from which the polymeric nanoparticles consist, but not in the core of the polymer chain.

Thus, the process corresponding to the present invention comprises two steps as a rule. In the first step, the production of the polymeric parent substance occurs in a known manner by subjecting vinyl monomers in aqueous dispersion medium to a emulsifier-free emulsion polymerization. In this connection, the vinyl monomers should have a low water solubility which should preferably amount to <20 g/l. Examples for such vinyl monomers are styrene, acrylic acid- or methacrylic acid derivatives. Preferable (meth)acrylic acid derivatives are alkyl(meth)acrylates (with an alkyl residue of 1 to 8 C-atoms) as well as N-alkyl or dialkylacrylamides, such as for example N-butylmethylacrylamide, N-isobutylmethylacrylamide or N-octylmethylacrylamide.

It is to be considered as inventive that the emulsion polymerization, which is preferably carried out at temperatures from 20 to 100° C., occurs without the addition of the emulsifiers and that the surface charge of the polymeric parent substance is induced alone by the use of ionic radical starters. In this connection, cationic initiators with basic end groups, such as for example 2,2′-azobis(2-amidinopropane)dihydrochloride (AIBA) or 2,2′-azobis(2-(2-imidazolin-2-yl)propane)dihydrochloride) (AIBI) have proven themselves above all.

As a result of the fact that no additional foreign material such as emulsifiers or stabilizers are added to the reaction, a complicated separation of these materials in a subsequent purification step is not required.

Since work is carried in aqueous dispersion medium, the production process of the polymeric parent compound is designed in a particularly cost effective manner. Preferably, the polymerization step is carried out in the form of a batch process whose up-scaling does not cause problems because fluctuations in temperature control as well as the selected stirring technique only have a small influence on the properties of the end product.

Depending on the selected monomer- and initiator concentration of the reaction batch, the polymeric particles have a particle size of preferably 10 to 1000 nm and a surface charge of 0.1 to 10 C/g polymer after the emulsion polymerization.

The obtained polymer suspensions are also storage-stable at room temperature over several months such that agglomerations of any type are not to be observed. The further advantages of these polymeric carrier materials are their high stability in biological media and low toxicity in the desired field of application.

According to a preferred embodiment, the polymer suspensions as well as the nucleic acids are purified for example by centrifugation or diafiltration before the reaction of the polymer suspensions. Customary dialysis processes and membranes can be used in the preferred diafiltration.

Additionally, it is possible within the scope of the present invention to add stabilizers in an amount of preferably 0.01 to 5% by weight with respect to the weight of the suspension to the polymer suspensions after the polymerization in order to achieve an additional steric stabilization of the polymer suspension in this manner. In this connection, biologically inert, non-ionic block co-polymers with hydrophobic and hydrophilic portions are preferably used as stabilizers. Example for such stabilizers are poloxameres or poloxamines

The actual reaction of the polymer suspensions with the nucleic acids corresponding to the second step of the process according to the invention preferably occurs at temperatures of 10 to 30° C. and a pH value <11, whereby the nucleic acids used for binding should be present in deprotinated form. In this connection, deoxyribonucleotides, ribonucleotides or chemically modified deoxyribonucleotides and ribonucleotides with 7 to 40 nucleotide units are considered as nucleic acids for example. However, plasmids can additionally be used as nucleic acids without any problem.

In a preferred embodiment, the polymeric nanoparticle-nucleic acid conjugates according to the invention arising from this reaction that have a certain negative excess charge due to the nucleic acid portion can be additionally modified in a type of sandwich complex with peptides, proteins with an isoelectric point of >7 or polyethylenimine. The loading capacity of the carrier material with nucleic acids and/or peptides or proteins is not influenced by the addition of steric stabilizers. The effectively bound nucleic acid and/or peptide or protein amounts can be easily determined by centrifugation of the polymeric nanoparticle-nucleic acid conjugates and measurement of the excess, unbound amount of substrate present in the supernatant.

The polymeric nanoparticle-nucleic acid conjugates according to the invention are excellently suited for gene transfer as well as for the control of gene expression as a result of their biological activity.

Rat hepatocytes, whose multiple drug resistance (mdr-) gene should be inhibited by the use of antisense oligonucleotides, serve as a test system for examining the efficiency of the polymeric nanoparticle-nucleic acid conjugates according to the invention. Surprisingly, the concentration of anti-mdr phosphorthioate oligionucleotides in the conjugates can be decreased by a factor of 10 in comparison to the free oligonucleotides in order to achieve the same effect. The difference when using of nuclease-labile phosphordiester oligonucleotides, which only exhibit an effect at all in the case of the conjugates according to the invention, because they are protected from enzymatic degradation in the conjugate form, is even more evident. However, as a result of their higher specificity and bio-compatibility, the unmodified, natural DNA fragments represent an interesting variant for an antisense strategy.

The polymeric nanoparticle-nucleic acid conjugates according to the invention in the form of plasmids are also excellently suited for gene transfer. For examining the loading capacity and effectiveness for gene transfer, plasmids are used which contain the β-galactosidase genes as easily detectable expression controls. The loading capacity (in μg DNA/mg polymer) was higher by a factor of approximately 4 than with short oligonucleotides fragments.

The particular advantages of the polymeric nanoparticle-nucleic acid conjugates according to the invention are essentially that neither the use of ionic co-monomers nor the use of hydrophobic cations as charge carriers is necessary for production because the negatively charged nucleic acids are electrostatically bound directly by the charged end groups to the particle surface. Side effects in biological systems, especially in cell culture experiments, which frequently arise when using biologically degradable carrier materials or adsorptively bound adjuvents are minimal in the present case because, aside from the bound nucleic acids and/or peptides, no biologically effective substances are released. As bio-compatibility tests on rat hepatocyctes demonstrate, the LDH release as an assay for the cytotoxicity for polystyrene at concentrations of 1 g polymer/l incubation medium, for example, was only about 5% higher after 20 hours than with the untreated control.

Thus, the polymeric nanoparticle-nucleic acid conjugates according to the invention and their complexes with basic peptides or proteins represent a new and efficient system for in vitro control of gene expression (anti-sense strategy). Additionally, they can be used in a broad manner as vectors for gene transfection. Thus, by varying the size, type and surface charge of the polymeric nano-particle parent substance by selection of the effective nucleic acid components and their derivation as well as by modification of the surface by means of basic peptides or polyethylenimine, the polymeric nanoparticle-nucleic acid systems can be adjusted to the respective requirements of the biological test systems.

The following examples more closely illustrate the invention.

### EXAMPLES

### Example 1

40 ml of purified water and 2.5 ml styrene, which was previously freshly purified under protective gas (argon), are added to a 100 ml round bottom flask provided with a KPG stirrer, reflux cooler, argon inlet and outlet devices and intensively intermixed under conduction of an argon stream. 50 mg 2,2′azobis(2-amidinopropane) dihydrochloride (AIBA), dissolved in 5 ml H₂O, are added to the batch and the reaction flask is immersed in an oil bath tempered to 80° C. and stirred at a stirring-rotation of 360 rpm. After approximately 10 minutes, a milky white clouding of the reaction batch is recognizable. After 24 hours, this is cooled to room temperature. A polymer suspension with a solids content of 42 g/l and particle diameter of 400 to 500 nm is obtained which has a considerably monodisperse size distribution of the particles. The material is dialysed against purified water over a period of 7 days by means of a dialysis membrane (SpectraPor; MWCO: 10000; Roth, Karlsruhe) in order to remove polymer dissolved in the dispersion medium and residual monomer traces. The purified product has a solids content of 40 g/l and a conductometrically determined particle charge of 759 mC/g polymer.

Phosphorothioate oligodeoxynucleotide 20mer having the sequence 5′ CCC TGC TCC CCC CTG GCT CC 3′ (DNA-1; M_{W}: 6207 g/mol) which is synthetically produced according to the phosphoramidite method is purified by means of preparative HPLC and, after cleaving the dimethoxytrityl protective group, is dialysed by means of a sterile dialysis membrane (SpectraPor CE; MWCO: 500; Roth, Karlsruhe) against purified water over a period of 7 days.

200 μl of the polymer suspension (8 mg polymer) are incubated with 100 ng (16 nmol) phosphorothioate oligonucleotide (DNA-1), dissolved in purified water, over a period of 12 to 24 hours. A polymeric nanoparticle-oligonucleotide conjugate with a nucleic acid portion of 1.7 μmol oligonucleotide/g polymer (10.6 mg nucleic acid/g polymer) is obtained.

The determination of the loading capacity occurs according to the following described method:

After incubation of the nano-particles with the nucleic acid, the conjugate is centrifuged (14000 g, 2×45 min.) and the amount of unbound substance in the supernatant is determined by means of UV measurement. The adsorptively bound amount of substance is calculated via the difference to the starting amount of nucleic acid and/or peptide.

### Example 2

The polymerization batch and the purification of the product occurs analogously to Example 1. However, in this connection, 1.5 ml of styrene is used. A polymer suspension is obtained with a solids content of 23 g/l. The particles have a particle diameter of 150 to 400 nm. After the dialysis, the solids content is 22 g/l and the particle charge is 1080 mC/g.

After incubation of 100 μl of the polymer suspension (2:2 mg polymer) with 23.8 ng (5.3 nmol) phosphordiester-oligonucleotide 15mer having the sequence 5′ TTC TTG TCT GCT CTT 3′ (DNA-2 M_{X}: 4491 g/mol) analogously to Example 1, a polymeric nanoparticle-oligonucleotide conjugate with a nucleic acid portion of 2.0 μmol oligonucleotide/g polymer (8.9 mg nucleic acid/g polymer) is obtained.

### Example 3

Analogous to Example 1, a solution of 118 mg 2,2′azobis(2-(2-imidazolin-2-yl)propane) dihydrochloride (AIBI) in 10 ml water is added to 80 ml H₂O and 3 ml styrene and polymerized over a period of 24 hours. The obtained polymer suspension has a solids content of 23.9 g/l and consists of particles with an average particle diameter of 150 to 200 nm that have a considerably monodisperse size distribution. After dialysis over a period of 21 days, the solids content is 22.7 g/l and the particle charge 930 mC/g. Since the polymer particles produced with AIBI have a higher surface charge and a higher loading capacity, such particles form the best conjugates with nucleic acids and therefore represent the best embodiment of the invention according to the present knowledge of the inventor.

The toxicity of the polymer suspension (stabilized with an aqueous solution of 0.1% (w/v) poloxamer 338 (ICI chemicals; Manchester, UK); density after stabilization: 9.56 g/l) in rat hepatocyctes, determined over the LDH release, is 81 units in comparison to 77 units of the untreated control (1 g polymer/l incubation medium) after 20 hours.

After incubation of 200 μl of the stabilized polymer suspension (4.5 mg polymer) with 72.7 ng (16.2 nmol) DNA-2 in a manner analogous to the method of Example 1, a polymeric nanoparticle-oligonucleotide conjugate is obtained with a nucleic acid portion of 3.0 μmol oligonucleotide/g polymer (13.5 mg nucleic acid/g polymer).

### Example 4

The incubation of 200 μl of the purified and stabilized suspension from Example 3 with 76.4 ng (16.2 nmol) of the phosphorothioate oligodeoxynucleotide DNA-3 (M_{W}: 4716 g/mol; sequence analogous to DNA-2 in Example 2) results in a polymeric nanoparticle-oligonucleotide conjugate with a nucleic acid portion of 3.1 μmol oligonucleotide/g polymer (14.6 mg nucleic acid/g polymer; determination analogous to Example 1).

The inhibition of mdr gene expression of the polymeric nanoparticle-DNA-3 conjugate is examined by means of mRNA determination (Northern Blot) and mdr protein determination (Western Blot) from rat hepatocyctes. The polymeric nanoparticle-oligonucleotide conjugates are used in concentrations of 0.032, 0.096, 0.32 and 0.96 g polymer/l incubation medium (0.1, 0.3, 1 and/or 3 μmol DNA-3/l incubation medium). At a concentration of 1 μmol DNA-3/l incubation medium, the mRNA content can be decreased by 90% and the protein content by 50%. With free oligonucleotide, the same inhibition effect is first achieved at a concentration of 10 μmol DNA-3/l incubation medium. Control oglionucleotides (DNA-4; sequence: 5′ CCT GTT GTT TTC TCT 3′ and/or DNA-5; sequence: 5′ AAG AGC AGA CAA GAA 3′) show no effect at all at the concentrations used in the free state or in the conjugate form with nanoparticles.

### Example 5

To test the batch size of the polymerization on the properties of the end product, the reaction batch of Example 3 was increased by five-fold. The characterisation of the purified material resulted in no noteworthy differences with respect to particle size, particle distribution, surface charge and loading capacity of nucleic acid to the results from Example 3 (solids content after the dialysis: 23.8 mg; particle charge: 1020 mC/g polymer; nucleic acid portion: 3.4 μmol DNA-3/g polymer and/or 16.0 mg DNA-3/g polymer; determination analogous to Example 1).

### Example 6

A polymeric nanoparticle-plasmid conjugate is obtained with the incubation of 50 μl of the stabilized polymer suspension from Example 3 with 22 μg pcMVβ plasmid dissolved in 100 μl purified water with a nucleic acid portion of 42 mg nucleic acid/g polymer. The conjugate is incubated for 12 to 24 hours with 2.6 μg (0.95 nmol) of the peptide penetratin (homeodomain-sequence; M_{W}: 2720.2 g/mol; Derossi, D., Joliot, A. H., Chassaing, G., Prochiantz; A., J. Biol. Chem. 1994, 269 (14), 10444-10450). The determination of the loading capacity of plasmid and/or peptide occurs analogously to Example 1.

### Example 7

The polymerization batch corresponds to that in Example 3, however, 2 ml styrene was used. The crude product possesses a solids content of 10.4 g/l and has a polydisperse distribution of the particle diameter of 50 to 200 nm. After purification by means of dialysis over a period of 10 days, the solids content is 8.2 g/l and the particle charge 2190 mC/g.

1. Process for the production of biologically active polymeric nanoparticle-nucleic acid conjugates by polymerization of vinyl monomers with a low water solubility in aqueous solution and subsequent reaction of the obtained polymer suspensions with the nucleic acids, characterized in that the polymerization of the vinyl monomers is carried out in the presence of cationic radical starters and in the form of an emulsifier-free emulsion polymerization.

2. Process according to claim 1, characterized in that the vinyl monomers have a water solubility of <20 g/l.

3. Process according to claims 1 and 2, characterized in that styrene, acrylic acid derivatives, methacrylic acid derivatives or mixtures thereof are used as vinyl monomers.

4. Process according to claims 1 to 3, characterized in that cationic radical starter with basic end groups, such as for example, 2,2′-azobis(2-amidinopropane) dihydrochloride (AIBA) or 2,2′azobis(2-(2-imidazolin-2-yl)propane) dihydrochloride (AIBI) are used.

5. Process according to claims 1 to 4, characterized in that the emulsion polymerization is carried out at temperatures from 20 to 100° C.

6. Process according to claims 1 to 5, characterized in that the polymer particles have a particle size of 10 to 1000 nm after the emulsion polymerization.

7. Process according to claims 1 to 6, characterized in that the polymer suspensions are purified before the reaction with nucleic acids, for example by centrifugation or by diafiltration.

8. Process according to claims 1 to 7, characterized in that the reaction of the polymer suspensions with the nucleic acids occurs at temperatures from 10 to 30° C. and a pH value <11.

9. Process according to claims 1 to 8, characterized in that deoxyribonucleotides, ribonucleotides or chemically modified deoxyribonucleotides and ribonucleotides with 7 to 40 nucleotide units are used as nucleic acids.

10. Process according to claims 1 to 8, characterized in that plasmids are used as nucleic acids.

11. Process according to claims 1 to 10, characterized in that the stabilizers in an amount of 0.01 to 5% by weight with respect to the weight of the suspension are added to the polymer suspensions after the polymerization.

12. Process according to claim 11, characterized in that non-ionic block copolymers with hydrophobic and hydrophilic portions are used as stabilizers.

13. Process according to claim 12, characterized in that poloxameres or poloxamines are used as non-ionic block-copolymers.

14. Process according to claims 1 to 13, characterized in that the polymeric nanoparticle-nucleic acid conjugates are modified with peptides, proteins with an isoelectric point >7 or polyethylenimine.

15. Polymeric nanoparticle-nucleic acid conjugates obtainable according to a method according to one of the proceeding claims.

16. Use of a polymeric nanoparticle-nucleic acid conjugates according to claim 15 for gene transfer.

17. Use of polymeric nanoparticle-nucleic acid conjugates according to claim 15 for the control of gene expression.
